(19) United States
(12) Patent Application Publication
(10) Pub. No.: US 2024/0293118 A1
(43) Pub. Date: Sep. 5, 2024

(54) COMPRESSION FASTENER, INSTRUMENTS AND METHODS

(71) Applicant: Medartis AG, Switzerland (CH)

(72) Inventors: Thomas HOLTON, Warsaw, IN (US); Adam Finley, Warsaw, IN (US)

(73) Assignee: Medartis AG, Switzerland (CH)

(21) Appl. No.: 18/581,887

(22) Filed: Feb. 20, 2024

Related U.S. Application Data
(60) Provisional application No. 63/487,888, filed on Mar. 2, 2023.

(51) Int. Cl. A61B 17/064; A61B 17/80

(52) U.S. Cl. CPC A61B 17/0642; A61B 17/8004; A61B 2017/0645

(57) ABSTRACT

Examples of the invention relate to a compression fastener and methods of use, including using an inserter to apply a pre-stress to the compression fastener, for compressing first and second bone portions or a bone portion together.

### CROSS-REFERENCED TO RELATED APPLICATION

The present application claims priority to U.S. Provisional Application Ser. No. 63/487,888 filed on Mar. 2, 2023 (Attorney Docket No. 3768.131P1), which is incorporated herein by reference in its entirety.

### FIELD OF THE INVENTION

Examples of the invention relate to methods, implants, and instruments and, more particularly, a compression fastener or staple for compressing bone portions together.

### BACKGROUND

Various conditions may affect skeletal joints such as the deterioration, elongation, shortening, or rupture of soft tissues, cartilage, and/or bone associated with the joint and consequent laxity, pain, and/or deformity. It may be desirable to change the angular alignment of a bone or a portion of a bone to restore function and/or reduce pain. It likewise may be desirable to fuse a joint to fix the bones of the joint in a better angular alignment or reduce pain caused by motion at the joint. It may also be desirable to support a fractured bone to allow healing of the fracture to occur. It may also be desirable to support an implant on a bone. To this end, various osteotomy procedures, joint fusion procedures, fracture fixation procedures, joint resurfacing procedures, implants and instruments have been proposed. Such procedures have been performed throughout the bridge to make various angular adjustments in, fuse joints associated with, fuse fractures associated with, and/or resurface articular surfaces of tibia, fibula, femur, pelvis, humerus, ulna, radius, carpal, metacarpal, tarsal, metatarsal, phalangeal, and other bones.

### SUMMARY

Examples of the invention provide methods, implants, including bone plates, and instruments capable of compressing bone portions or a bone portion and an implant together. The bone portions may be portions of the same bone as in a fracture or osteotomy. The bone portions may be portions of different bones as in arthrodesis. A bone portion may be a portion of a bone adjacent an articulating joint and the implant may be a resurfacing implant, a spacer, and/or a fusion supporting implant.

In one embodiment, a bone fastener device that comprises a first post, a second post and a bridge extending between the first post and the second post. The bridge is configured to be stretched or extended by a pre-stress applied by, for example, an inserter, prior to insertion into bone. In one example, the bridge may be made from a biocompatible super elastic metal. Once bone fastener is inserted into the bone, the pre-stress applied to the fastener is removed to allow the first and second posts to move towards each other, causing the bone to compress.

In one embodiment, the bridge may include a first rib, a second rib and a central portion extending between the first rib and the second rib. The first and second rib may arcuately extend laterally between the first post and the second post. In one example, the central portion may be circular in shape and include a center aperture. The center aperture may be configured to receive, for example, a cross fixation screw. However, the bridge may be constructed in different ways and/or shapes to allow the first and second posts to flex apart.

### BRIEF DESCRIPTION OF THE DRAWINGS

Various examples of the invention will be discussed with reference to the appended drawings. These drawings depict only illustrative examples of the invention and are not to be considered limiting of its scope.

FIG. 1A depicts a perspective view of one embodiment of a bone fastener constructed in accordance with one or more aspects of the present invention;

FIG. 1B depicts a front view of the bone fastener depicted in FIG. 1A;

FIG. 1C depicts a top view of the bone fastener depicted in FIG. 1A;

FIG. 1D depicts a bottom view of the bone fastener depicted in FIG. 1A;

FIG. 1E depicts a side view of the bone fastener depicted in FIG. 1A;

FIG. 2A depicts a front view of one embodiment of an inserter constructed in accordance with one or more aspects of the present invention;

FIG. 2B depicts a perspective view of one embodiment of the inserter depicted in FIG. 2A coupled to a fastener depicted in FIG. 1A;

FIG. 3 depicts one example of a fastener 300A in a relaxed state and a fastener 300B in a pre-stressed state from, for example, use of an inserter constructed in accordance with one or more aspects of the present invention;

FIG. 4A depicts a front view of another embodiment of an inserter constructed in accordance with one or more aspects of the present invention; and

FIG. 4B depicts a perspective view of one embodiment of the inserter depicted in FIG. 4A coupled to a fastener depicted in FIG. 1A.

### DESCRIPTION OF THE ILLUSTRATIVE EXAMPLES

The following illustrative examples describe methods, implants, and instruments capable of compressing first and second bone portions or a bone portion and an implant together and/or fixing fractures by, for example, reducing the fracture and preventing any movement, while also shielding the fracture site from stress to allow healing. The bone portions may be portions of the same bone that have become separated due to a fracture or an osteotomy. The bone portions may be portions of different bones as in an arthrodesis performed to fuse a joint. A bone portion may be a portion of a bone adjacent an articulating joint and the implant may be a resurfacing implant, a spacer, and/or a fusion supporting implant. Examples of the invention may be used with any bone or joint including but not limited to bones such as a tibia, fibula, femur, pelvis, humerus, ulna, radius, carpal, metacarpal, tarsal, metatarsal, phalange and joints associated therewith.

The term “transverse” is used herein to mean crossing as in non-parallel.

Examples according to the invention provide methods, implants, and instruments capable of compressing first and second bone portions or a bone portion and an implant together.

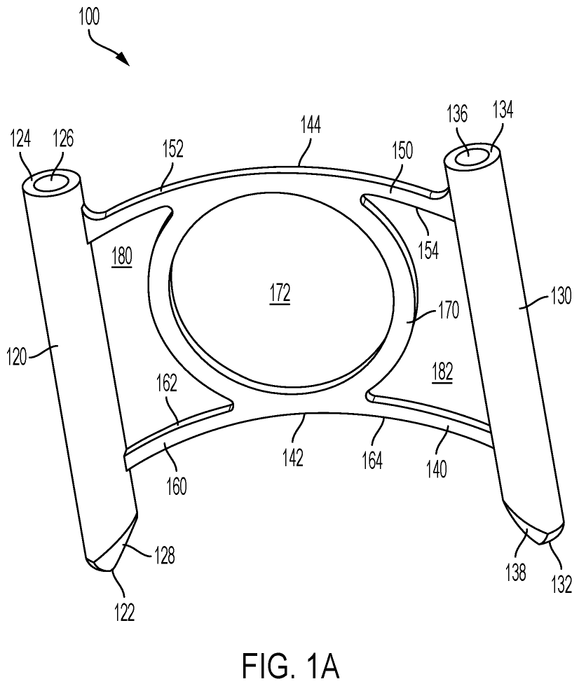

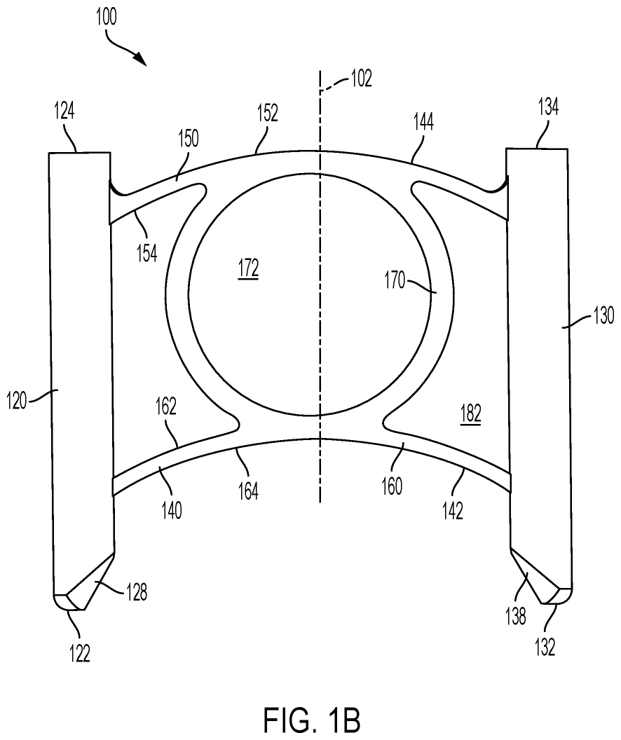

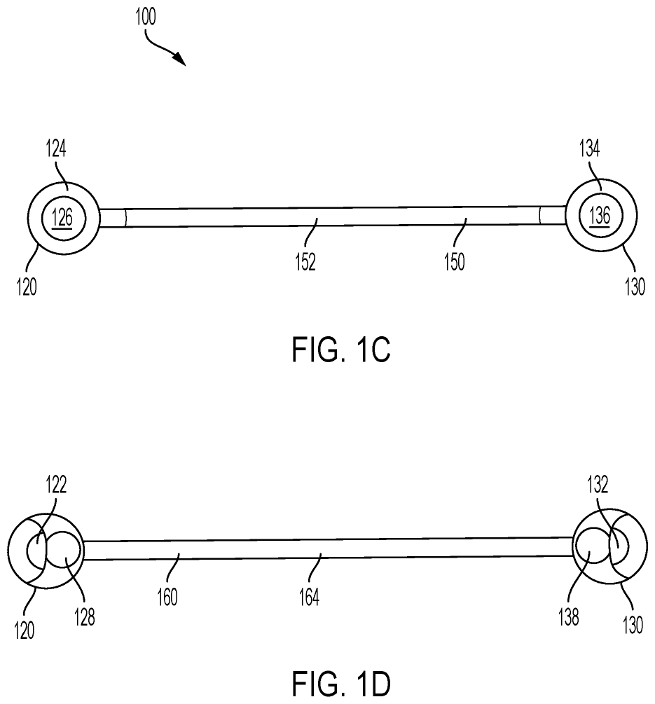

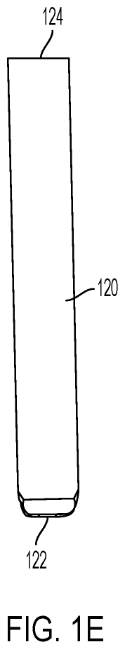

FIGS. 1A-1E illustrate an example in the form of a fastener 100 for joining first and second bone portions constructed in accordance with one or more aspects of the present invention. Fastener includes an insertion axis 102 along which fastener 100 moves as it is inserted into or removed from a bone. Fastener 100 includes first and second posts 120, 130 connected to a bridge 140.

First and second posts 120, 130 may be the same size or they may be different sizes to accommodate particular anatomy. For example, first and second posts 120, 130 may have the same width and depth but have different lengths so that they can accommodate bi-cortical fixation in bone portions of varying thickness. In the illustrative example of FIGS. 1A-1E, each post 120, 130 includes a generally cylindrical cross-section and extend from a leading end 122, 132 to a trailing end 124, 134, respectively.

As illustrated in FIGS. 1A and 1C, trailing ends 124, 134 of each post 120, 130, respectively, includes a cavity 126, 136 operable to couple with, for example, an inserter 200, 400 as described below. Preferably, cavities 126, 136 are threaded to receive a threaded bolt of an inserter as described below. In one example, leading ends 122, 132 of each post 120, 130 may include a radius 128, 138 to ease insertion of fastener 100 into holes formed in bone.

The various sizes and proportions for fastener 100 will vary based on the application. For example, depending on the application, post depth preferably ranges from 2 mm to 7 mm, and the bridge thickness preferably ranges from 0.5-5 mm. Further for example, in many applications, such as for use in the mid and fore regions of the hands and feet, a fastener may advantageously have a post depth of 2.5-4.5 mm and a bridge thickness of 0.5-1.5 mm. The ratio of post depth to bridge thickness preferably ranges from 14:1 to 1.5:1. More preferably, the ratio ranges from 5:1 to 3:1.

First and second posts 120, 130 may be the same length or different lengths, may be staggered at one or both ends, or the post lengths are the same and the posts are level at the proximal end but staggered at the distal end. For use in foot surgery, the post lengths are preferably in the range of 10-50 mm and more preferably in the range of 14-32 mm. For use at other locations, the post length may be outside of these ranges and can be, for example, quite long in large staples for applications such as tibial osteotomies.

Bridge 140 extends between first post 120 and second post 130 and includes a distal or leading end 142 and a proximal or trailing end 144. Leading end 142 and trailing end 144 are spaced from one another longitudinally relative to insertion axis 102. In one embodiment, bridge 140 may be stressed to widen the lateral distance between first and second posts 120, 130 prior to insertion and then, once fastener 100 is inserted into the bone, that pre-stress is removed to then compress the bones together. In one embodiment, bridge 140 may be made from a biocompatible super elastic metal, such as, for example, nickel titanium alloy, such as Nitinol, or other titanium alloys.

In the illustrative example of FIGS. 1A-1B, the bridge 140 includes a top rib 150, a bottom rib 160 and a connecting portion 170 extending between top rib 150 and bottom rib 160. Both top rib 150 and bottom rib 160 extend laterally and arcuately between first post 120 and second post 130 to provide stable rotation and stability. Top rib 150 includes a top surface 152 having a convex shape and a bottom surface 144 having a concave shape. Bottom rib 160 includes a top surface 162 having a convex shape and a bottom surface 164 having a concave shape. Bottom surface 154 of top rib 150 is spaced from top surface 162 of bottom rib 160, creating spaces 180, 182 between bottom surface 154 of top rib 150, top surface 162 of bottom rib 160, connecting portion 170 and first post 120 and second post 130. As a force, stress or pressure is applied to bridge 140 prior to insertion into bone, as will be described in more detail below, top surface 152 of top rib 150 becomes less convex and bottom surface 164 of bottom rib 160 become less concave resulting into or creating more distance between first post 120 and second post 130 as top rib 150 and bottom rib 160 become straighter.

Connecting portion 170 may be shaped in a number of ways, including, but not limited to, a circle (as illustrated in FIGS. 1A-1B), a fish mouth, an ellipse, a rhombus, or other shapes. The shape and elastic nature of bridge provides a “balanced” compression between top and bottom of implant posts 120, 130 when a force, stress or pressure is applied to bridge 140 prior to insertion into bone, as will be described in more detail below.

In the illustrative example of FIGS. 1A-1B, connecting portion 170 of bridge 140 may form a central aperture 172. The shape and size of central aperture 172 may be determined for the particular application in which the fastener 100 is to be used. For example, the aperture may receive a fixation member, such as a screw member, to provide cross fixation of the bone portions and to prevent the fastener 100 from migrating out of the bone.

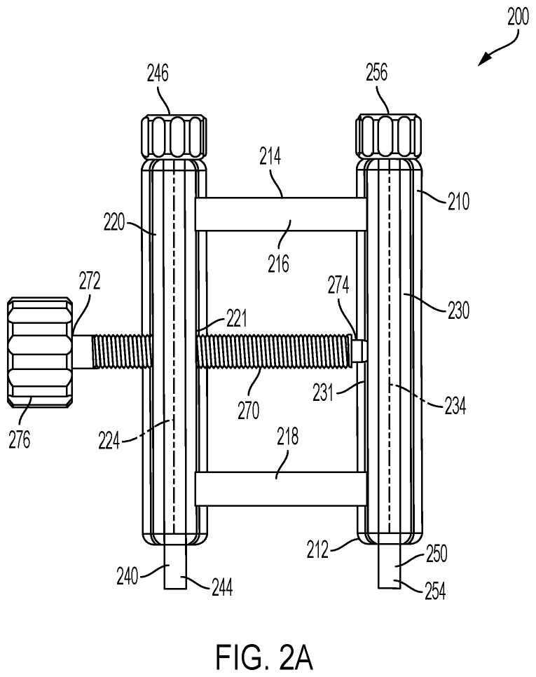

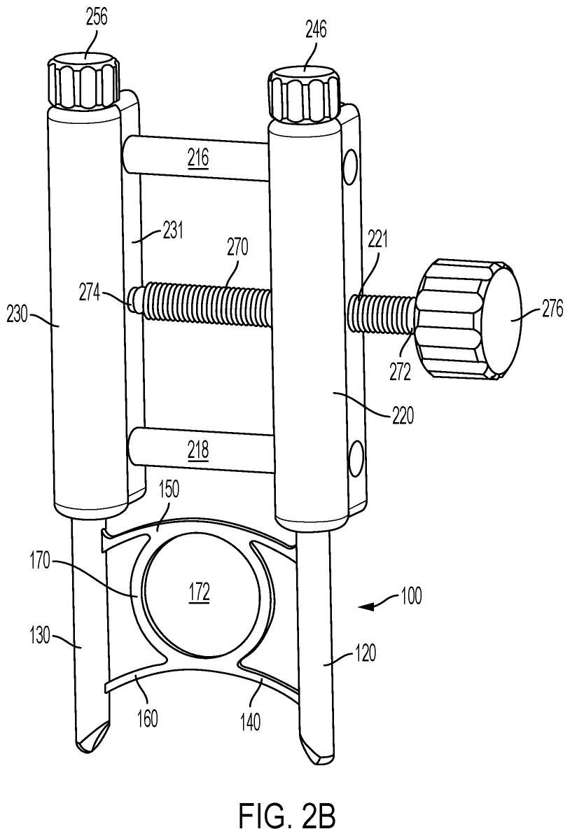

FIG. 2A depicts one embodiment of an inserter 200 constructed in accordance with one or more aspects of the present invention. Referring to FIG. 2A, inserter 200 is configured for use with the fastener 100 of FIGS. 1A-1E. Inserter 200 includes a body 210, two locking bolts 240, 250 and a bridge adjusting bolt 270. Body 210 includes a distal end 212 and a proximal end 214. Body 210 includes a first member 220 and a second member 230 laterally spaced from first member 220. First member 220 and second member 230 may be connected by, for example, two lateral members 216, 218 extending between first and second members 220, 230. First and second members 220, 240 each include a passage (not shown) extending from distal end 212 to proximal end 214. Each passage defines a passage axis 224, 234 parallel to the insertion axis 102 that, in use, align with threaded cavities 126, 136 of fastener 100. Each passage receives a locking bolt 240, 250 in axial and rotating relationship. Each bolt 240, 250 includes a first portion 242, 252 exposed beyond distal end 212 of a laterally spaced members 220, 230 and a second portion 244, 254 exposed beyond proximal end 214 of a laterally spaced first and second members 220, 230. A knob 246, 256 is fixed to first portions 242, 252 of each bolt 240, 250 to allow a user to rotate bolts 240, 250 about the passage axis 224, 228. Second portion 244, 254 of each bolt 240, 250 is threaded and sized to screw into threaded cavity 126, 136 in first and second posts 120, 130.

Bridge adjusting bolt 270 includes a first end 272 and a second end 274. A knob 276 is fixed to first end 272. Bridge adjusting bolt 270 extends from knob 276, through a threaded hole 221 in first member 220, towards second end 274. Second end 274 abuts against an interior surface 231 of second member 230 facing first member 220. Knob 276 allows a user to rotate bridge adjusting bolt 270 to adjust the lateral distance between first member 220 and second member 230, which, in use, adjusts the lateral distance between first post 120 and second post 130 when inserter 200 is coupled to fastener 100 as described in more detail below.

As illustrated in FIG. 2B, inserter 200 may be removably joined or coupled to fastener 100 by first aligning locking bolts 240, 250 proximally to cavities 126, 136 in each first and second post 120, 130. Threaded portion 244, 254 of each locking bolt 240, 250 may then be inserted into threaded cavity 126, 136 of fastener 100. Each knob 246, 256 is then rotated to thread locking bolts 240, 250 into threaded cavity 126, 136 and secure fastener 100 to inserter 300 as shown in FIG. 2B. After locking bolts 240, 250 secure inserter 200 to fastener 100, a user may then turn knob 276 connected to bridge adjusting bolt 270 to separate by, for example, pulling first post 120 laterally away from second post 130 of fastener 100, which will, in turn, put a pre-stress on bridge 140 of fastener 100 to increase the lateral distance between posts 120, 130 of fastener 100. Once a desired pre-stress has been achieved by use of bridge adjusting bolt 270, fastener 100 may be inserted into bone.

Once fastener 100 is inserted into the bone, inserter 200 is removed and fastener 100 will compress the bone between first and second posts 120, 130 uniformly along posts as bridge 140 de-stresses and the lateral distance between first and second posts 120, 130 decreases. In other words, after insertor 200 is removed, the pre-stress to bridge 140 by inserter 200 is removed and the bone is compressed between fastener posts 120, 130 normal to the insertion direction or insertion axis 102. The amount of compression can be tailored by pre-stress applied to fastener 100 by inserter 200 prior to insertion into bone.

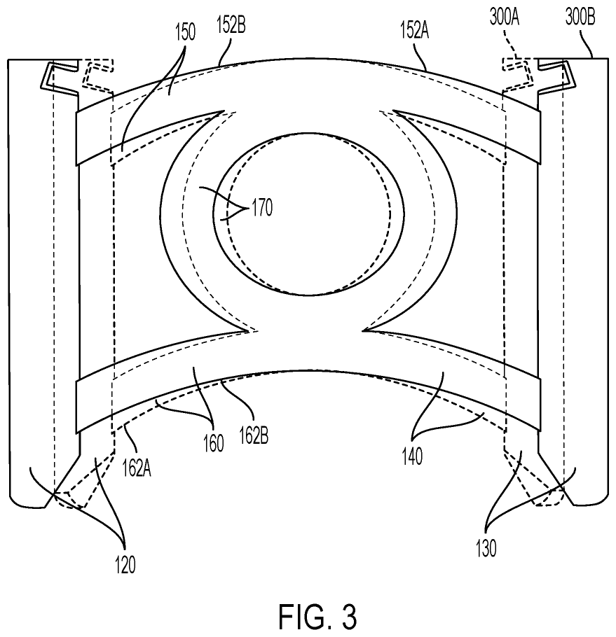

FIG. 3 depicts a fastener 300A in a relaxed state prior to use of, for example, inserter 200 illustrated in FIGS. 2A-2B, and fastener 300B in a pre-stressed state in which bridge 140 is expanded or stretched laterally using, for example, inserter 200. As shown, top rib 150 and bottom rib 150 of bridge 140 are stretched out as a result from the force applied by, for example, inserter 200 where top surface 152B of pre-stressed top rib 150B is less convex than top surface 152A of relaxed state top rib 150A, and bottom surface 160B of pre-stressed bottom rib 160B is less concave than bottom surface 160A of relaxed state bottom rib 160A.

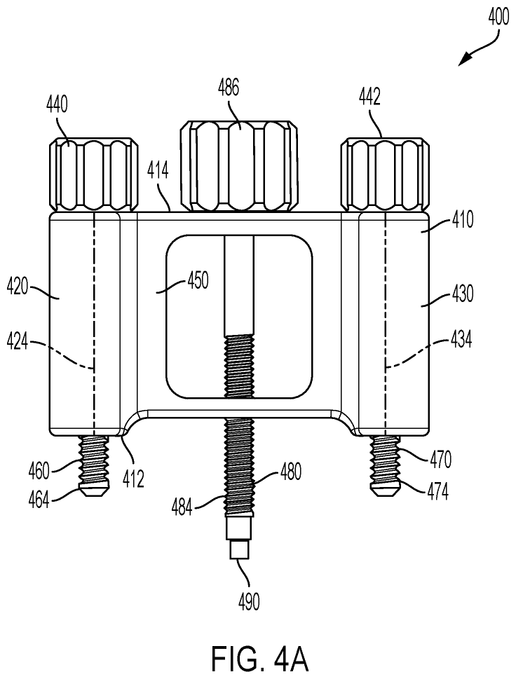

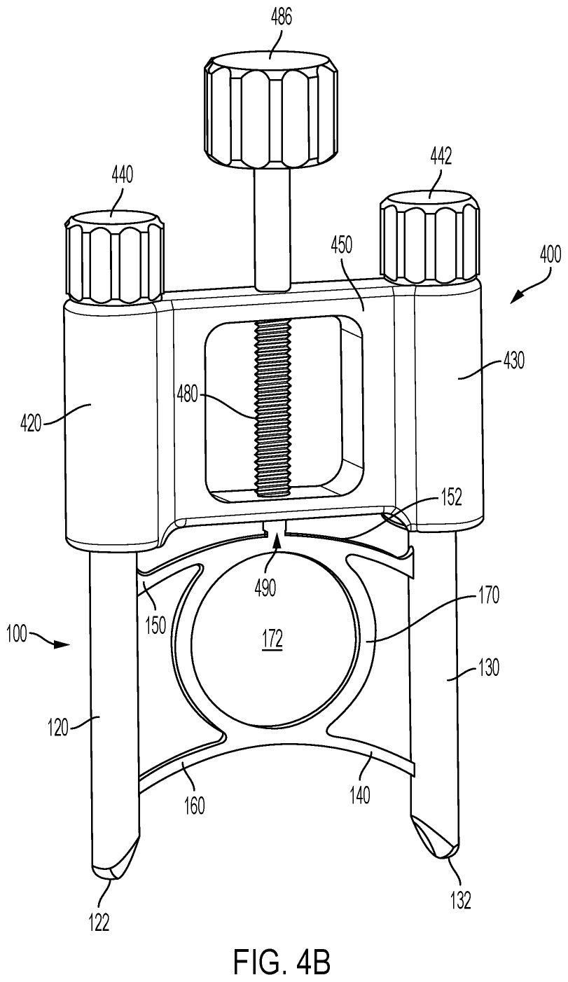

FIG. 4A illustrates another embodiment of an inserter 400 constructed in accordance with one or more aspects of the present invention. In this example, inserter 400 includes a body 410, two locking bolts 460, 470 and a bridge adjusting bolt 480. Body 410 includes a distal end 412 and a proximal end 414. Body 410 includes a first member 420, a second member 430 laterally spaced from first portion 420, and a middle portion 450 between first and second members 420, 430. First and second members 420, 430 each include a passage 422, 432 extending from distal end 412 to the proximal end 414. Each passage 422, 432 defines a passage axis 424, 434 parallel to the insertion axis 102 that, in use, align with threaded cavities 126, 136 of fastener 100. Each passage 422, 432 receives a locking bolt 460, 470 in axial and rotating relationship. Each bolt 460, 470 includes a first portion (note shown) exposed beyond distal end 412 of first and second members 420, 430 and a second portion 464, 474 exposed beyond proximal end 414 of a laterally first and second members 420, 430. A knob 440, 442 is fixed to first portion of each bolt 460, 470 to allow a user to rotate bolts 460, 470 about the passage axis 424, 434. Second portion 464, 474 of each bolt 460, 470 is threaded and sized to screw into threaded cavity 126, 136 in one of the fastener posts 120, 130.

As illustrated in FIG. 4A, bridge adjusting bolt 480 extends through middle portion 450 of body 410 from proximal end 414 to distal end 412 of inserter 400. Bridge adjusting bolt 480 includes a first portion (not shown) exposed beyond proximal end 414 of middle portion 450 and a second portion 484 exposed beyond distal end 412 of middle portion 450. A knob 486 is fixed to first portion of bridge adjusting bolt 480. Second portion 484 extends through distal end 412 of inserter body 410 and includes a tip 490 configured or designed to engage or abut against, for example, bridge 140 of fastener 100. In one example, knob 486 allows a user to rotate bridge adjusting bolt 480 and apply a force through tip 490 to top surface 152 of top rib 150 of bridge 140 of fastener 100. As knob 486 rotates and tip 490 of bridge adjusting bolt 480 applies a force to top surface 152 of top rib 150 to flatten bridge 140 or reduce the arc of top rib 150 and bottom rib 160, bridge 140 expands laterally, transverse to insertion axis 102, causing posts 120, 130 of fastener 100 to laterally separate from each other.

As illustrated in FIG. 4B, inserter 400 is joined to the fastener 100 by first aligning the locking bolts 460, 470 proximally to cavities 126, 136 in each post 120, 130. Threaded second portion 464, 474 of each locking bolt 460, 470 may then be inserted into threaded cavity 126, 136 of fastener 100. Each knob 440, 442 is then rotated to thread locking bolts 460, 470 into threaded cavity 126, 136 and secure fastener 100 to inserter 400. After locking bolts 460, 470 secure inserter 200 to fastener 100, a user may turn knob 486 connected to bridge adjusting bolt 480 to apply a desired force to bridge 140 from tip 490, which will, in turn, put a pre-stress on bridge 140 of fastener 100 to increase the distance between posts 120, 130 of fastener 100. Once a desired pre-stress has been achieved by use of bridge adjusting bolt 480, fastener 100 may be inserted into bone.

Once fastener 100 is inserted into the bone, inserter 400 is removed and fastener 100 will compress the bone between fastener posts uniformly along fastener posts 120, 130 as bridge 140 de-stresses and the lateral distance between posts 120, 130 decreases. In other words, after insertor 400 is removed, the pre-stress to bridge 140 by inserter 400 is removed and the bone is compressed between fastener posts 120, 130 normal to the insertion direction. The amount of compression can be tailored by the pre-stress applied to fastener 100 by inserter 400 prior to insertion into bone.

Preferably the fastener 100 is seated with trailing ends 124, 134 of fastener posts 120, 130 and trailing end 144 of the bridge 140 flush with or below the bone surface to reduce irritation of surrounding tissues. To remove the fastener, it is pulled proximally. T r ailing edge 144 of the bridge 140 aids in passing bridge 140 through any bone that has grown over the bridge 140.

In one embodiment one or more bone plates may be coupled to threaded cavities 126, 136 after fastener 100 is inserted into bone. The one or more bone plates may be used to, for example, secure fastener 100 in place while fastener 100 compresses the bone.

The implants, instruments and methods of examples of the invention may be used at many different locations within a patient to secure bone portions relative to one another and may further be used to form various constructs, including, but not limited to, a human foot, including, for example, metatarsophalangeal, metatarsocuneiform or phalangeal fusion, calcaneal osteotomy, a human hand, including, for example, phalangeal fusion, metacarpophalangeal fusion, midshaft fusion, metacarpocarpal fusion, and carpal fusion, closing wedge tibial osteotomy. While illustrative, these examples are not comprehensive and it will be apparent to one skilled in the art that these implants, instruments, and methods may be used anywhere two bone portions are to be secured. The size and proportion of the fastener may be varied to suit a particular anatomical location.

While several aspects of the present invention have been described and depicted herein, alternative aspects may be affected by those skilled in the art to accomplish the same objectives. Accordingly, it is intended by the appended claims to cover all such alternative aspects as fall within the true spirit and scope of the invention.

What is claimed:

1. A bone fastener system, said bone fastener system comprising:
a first post;
a second post; and
a bridge extending between said first post and said second post, said bridge made of a biocompatible super elastic metal, said bridge configured to expand laterally to change a distance between said first post and said second post from a first lateral distance to a second lateral distance from each other.

2. A bone fastener, said bone fastener comprising:
a first post;
a second post, said second post positioned laterally from said first post;
a bridge extending arcuately between said first post and said second post, said bridge made of a biocompatible super elastic metal and configured to change a distance between said first post and said second post responsive to a force applied to said bridge.

3. A bone fastener of claim 2, wherein said bridge including a first rib, a second rib and a center portion extending between the first rib and the second rib.

4. The bone fastener of claim 2, wherein the center portion includes a circular shape.

5. The bone fastener system of claim 2, wherein the first rib includes a top surface and a bottom surface, wherein the top surface is convex shaped.

6. The bone fastener of claim 5, wherein the second rib includes a top surface and a bottom surface, wherein the bottom surface is concave shaped:

7. A bone fastener system, said bone fastener system comprising:
a bone fastener, said bone fastener comprising:
a first post;
a second post, said second post positioned laterally from said first post;
a bridge extending between said first post and said second post, said bridge made of a biocompatible super elastic metal, said bridge configured to expand laterally to change a distance between said first post and said second post from a first lateral distance to a second lateral distance from each other; and
an inserter, said inserter including a body comprising first member and a second member, the first member configured to couple to said first post and the second member configured to couple to said second post, wherein said inserter configured to apply a force to said bridge to expand said bridge laterally change the distance between said first post and said second post from the first lateral distance to the second lateral distance from each other.

8. A method of compressing a first bone portion and a second bone portion together, said method comprising:
providing a bone faster, said bone fastener including a first post and a second post and a bridge extending between said first post and said second post, said second post positioned a lateral distance from said first post, said bridge made of a biocompatible super elastic metal, said bridge configured to expand laterally to change a distance between said first post and said second post from a first lateral distance to a second lateral distance from each other;
providing an inserter, said inserter including a body comprising a first member and a second member, the first member laterally spaced from the second member, said inserter include an adjusting bridge member;
removably coupling the first member of said inserter to said first post;
removably coupling the second member of said inserter to said second post;
engaging the adjusting bridge member of said inserter to apply a pre-stress to said bride by expanding said bridge to change the lateral distance between said first post and said second port from the first lateral distance to the second lateral distance from each other;
preforming a first hole from a surface of the first bone portion distally into the first bone portion;
preforming a second hole from a surface of the second bone portion distally into the second bone portion;
inserting said bone fastener with said first post in the first hole and said second post in the second hole; and
decoupling the first member from said first post and the second member from the second post to allow the lateral distance between the first post and the second post to change from the second lateral distance to the first lateral distance.
